(19) United States
(12) Patent Application Publication
(10) Pub. No.: US 2014/0038835 A1
(43) Pub. Date: Feb. 6, 2014

(54) METHODS FOR DIAGNOSING HYPERTROPHIC CARDIOMYOPATHY

(71) Applicants: Gordon Huggins, Bedford, MA (US); Martin Maron, Boston, MA (US)

(72) Inventors: Gordon Huggins, Bedford, MA (US); Martin Maron, Boston, MA (US)

(21) Appl. No.: 13/816,411

(22) PCT Filed: Aug. 12, 2011
(86) PCT No.: PCT/US11/47524

§ 371 Date: Sep. 23, 2013

Related U.S. Application Data
(60) Provisional application No. 61/373,545, filed on Aug. 13, 2010.

(51) Int. Cl. C12Q 1/68

(52) U.S. Cl. CPC C12Q 1/6883
USPC 506/9; 435/6.11; 536/24.31; 536/24.33; 506/16

(57) ABSTRACT

The present invention features a method for diagnosing hypertrophic cardiomyopathy by detecting one or more single nucleotide polymorphisms (SNPs) of the formin homology 2 domain containing 3 gene (FHOD3).

### BACKGROUND OF THE INVENTION

In general, the invention relates to methods for diagnosing hypertrophic cardiomyopathy (HCM) by detecting polymorphisms of the formin homology 2 domain containing 3 gene (FHOD3).

HCM is a disease of the myocardium in which a portion of the myocardium is hypertrophied. Genetic studies have identified HCM-causing mutations affecting the contractile apparatus, which includes the myosin heavy chain, myosin light chain, troponin, myosin binding protein C, and actin families of genes. These findings support a model by which a hypercontractile sarcomere induced by such genetic mutations is the proximal biophysical event that triggers a remodeling response, resulting in the HCM phenotype. Medical and surgical treatments for HCM provide only palliative benefits. Thus, understanding the genetic contributors to the variable HCM expressivity may help identify prognostic markers for HCM patients.

There exists a need in the art for additional methods for diagnosing hypertrophic cardiomyopathy.

### SUMMARY OF THE INVENTION

The present invention features a method for diagnosing hypertrophic cardiomyopathy by detecting one or more single nucleotide polymorphisms (SNPs) of the formin homology 2 domain containing 3 gene (FHOD3).

In one aspect, the invention features a method for identifying a subject with an increased risk for developing hypertrophic cardiomyopathy (HCM) by detecting in a biological sample obtained from the subject at least one SNP at the genomic locus of an FHOD3 gene, wherein the presence of at least one SNP at the genomic locus of the FHOD3 gene identifies the subject as having an increased risk for developing HCM. In another aspect, this method may be used to diagnose a subject as having HCM. In certain embodiments, the SNP may include rs516514.

The biological sample of the invention may include nucleic acid (e.g., DNA, genomic DNA, RNA, cDNA, hnRNA, or mRNA), and the nucleic acid may be extracted from the biological sample and amplified. In certain embodiments, the biological sample is obtained from heart tissue.

The detection step of the methods of the present invention may include or more of oligonucleotide microarray analysis, allele-specific hybridization, allele-specific polymerase chain reaction (PCR), 5′ nuclease digestion, molecular beacon assay, oligonucleotide ligation assay, size analysis, or nucleic acid sequencing.

In preferred embodiments of the invention, the subject is human. The subject may have a personal or family history of heart disease.

In another aspect, the invention features a kit that includes an assay for detecting at least one SNP at the genomic locus of an FHOD3 gene in a biological sample obtained from a subject, wherein the presence of at least one SNP at the genomic locus of said FHOD3 gene identifies the subject as having an increased risk for developing HCM. The assay of the kits may include nucleic acid probes and/or primers specific to the SNP(s) at the genomic locus of the FHOD3 gene and may additionally include instructions for correlating assay results with a subject's risk for having or developing HCM. In certain embodiments, the SNP may include rs516514.

In a final aspect, the invention features a microarray that includes oligonucleotide probes capable of hybridizing under stringent conditions to one or more nucleic acid molecules having a SNP at the genomic locus of an FHOD3 gene (e.g., the rs516514 SNP).

By “allele” or “allelic variant” is meant a polynucleotide sequence variant of a gene of interest. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other by a single nucleotide or several nucleotides and such differences can include substitutions, deletions, and insertions of nucleotides. An allele of a gene can also be a form of a gene containing a mutation.

By an “amplified” nucleic acid is meant to increase the number of copies of a single or few copies of, e.g., DNA or fragment thereof across several orders of magnitude, generating thousands to millions of copies of a particular DNA sequence. Methods for amplification include polymerase chain reaction (PCR), ligation amplification (or ligase chain reaction (LCR)), or any other amplification method known to one of skill in the art.

By “formin homology 2 domain containing 3” or “FHOD3” is meant a polynucleotide having the genomic nucleic acid sequence of NCBI Reference Sequence: NC_{—}000018.9 and the mRNA sequence of NCBI Reference Sequence: NM_{—}025135.2 or a polypeptide having the amino acid sequence of NCBI Reference Sequence: NP_{—}079411.2.

By “genotype” is meant the alleles of a gene contained in an individual or a sample. In the context of this invention, no distinction is made between the genotype of an individual and the genotype of a sample originating from the individual.

By “hypertrophic cardiomyopathy,” “HCM,” or “HOCM” is meant a disease of the myocardium in which a portion of the myocardium is hypertrophied (i.e., thickened). A diagnosis of hypertrophic cardiomyopathy may be made based on echocardiography, cardiac catheterization, cardiac magnetic resonance imaging (MRI), and genetic test findings, and family history of HCM or unexplained sudden death in otherwise healthy individuals.

By “increased risk” is meant that a subject is identified as being at a higher risk of, for example, hypertrophic cardiomyopathy due to the presence of one or more risk factors of HCM (e.g., the presence of a SNP of the FHOD3 gene). In one example, a subject's risk of developing HCM may be increased by at least 10%, 20%, 30%, 40%, 50%, 60%, 70%, 75%, 80%, 85%, 90%, 95%, or more, due to the presence of a SNP of the FHOD3 gene.

By “microarray” is meant an arrayed series of microscopic spots of oligonucleotides, each spot containing a specific nucleic acid sequence (e.g., a probe sequence). The specific nucleic acid sequences can be a short section of a gene or other nucleic acid element that is used to hybridize a nucleic acid sample (e.g., a target sample) under high-stringency conditions. In one embodiment, the microarray includes polynucleotides representative of FHOD3 nucleic acid sequences (e.g., FHOD3 SNP sequences).

By “polymorphic” or “polymorphism” is meant the occurrence of two or more genetically determined alternative sequences of a gene in a population. The polymorphic region or polymorphic site refers to a region of the polynucleotide where the nucleotide difference that distinguishes the variants occurs. Typically, the first identified allelic form is arbitrarily designated as the reference form and other allelic forms are designated as alternative or variant alleles. The allelic form occurring most frequently in a selected population is sometimes referred to as the wild-type form.

By “polynucleotide” or “nucleic acid” is meant any polyribonucleotide or polydeoxyribonucleotide, which may be unmodified RNA or DNA or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, hybrid molecules comprising DNA and RNA that may be single-stranded, or, more typically, double-stranded or a mixture of single- and double-stranded regions. In addition, polynucleotide refers to triple-stranded regions comprising RNA or DNA or both RNA and DNA. Nucleic acids may include, without limitation, mRNA, tRNA, rRNA, tmRNA, miRNA, siRNA, piRNA, aRNA, snRNA, snoRNA, shRNA, cDNA, msDNA, and mtDNA.

The term polynucleotide also includes DNAs or RNAs containing one or more modified bases and DNAs or RNAs with backbones modified for stability or for other reasons. Modified bases include, for example, tritylated bases and unusual bases such as inosine. A variety of modifications can be made to DNA and RNA; thus, polynucleotide embraces chemically, enzymatically, or metabolically modified forms of polynucleotides as typically found in nature, as well as the chemical forms of DNA and RNA characteristic of viruses and cells. Polynucleotide also embraces short nucleic acid chains, often referred to as oligonucleotides.

By “sample” is meant solid and fluid samples. By “biological sample” is meant cells (e.g., cardiomyocytes), protein or membrane extracts of cells, or blood, or biological fluids including, e.g., ascites fluid or brain fluid (e.g., cerebrospinal fluid (CSF)). Examples of solid biological samples include samples taken from feces, the rectum, central nervous system, bone, breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, and the thymus. Examples of biological fluid samples include samples taken from the blood, serum, CSF, semen, prostate fluid, seminal fluid, urine, saliva, sputum, mucus, bone marrow, lymph, and tears. Samples may be obtained by standard methods including, e.g., venous puncture and surgical biopsy. In certain embodiments, the biological sample is a heart, breast, lung, colon, or prostate tissue sample obtained by needle biopsy.

By “single-nucleotide polymorphism” or “SNP” is meant a polynucleotide that differs from another polynucleotide by a single nucleotide exchange. For example, exchanging one adenine (A) for one cytosine (C), guanine (G), or thymine (T) in the entire sequence of polynucleotide constitutes a SNP. Single-nucleotide polymorphisms may occur in coding regions (e.g., protein-coding regions) of genes, non-coding regions of genes, or in the intergenic regions between genes. SNPs within a coding sequence will not necessarily change the amino acid sequence of the protein that is produced due to degeneracy of the genetic code. SNPs that are not in coding regions may still have consequences for gene splicing, transcription factor binding, or the sequence of non-coding RNA.

By “stringent conditions” is meant a condition under which a specific nucleic acid hybrid is formed and non-specific nucleic acid hybrid is not formed. Stringency can be modified by, for example, modifying temperature and/or salt concentration. Detection of specific nucleic acid sequences with moderate or high similarity to, for example, an oligonucleotide probe depends on the stringency of the hybridization conditions. High stringency, such as high hybridization temperature and low salt in hybridization buffers, permits only hybridization between nucleic acid sequences that are highly similar, whereas low stringency, such as lower temperature and high salt, allows hybridization when the sequences are less similar

By “subject” is meant any animal, e.g., a mammal (e.g., a human). Other animals that can be diagnosed using the methods of the invention include, e.g., horses, dogs, cats, pigs, goats, rabbits, hamsters, monkeys, guinea pigs, rats, mice, lizards, snakes, sheep, cattle, fish, and birds.

By “target sequence” or “target region” is meant a region of a nucleic acid that is to be analyzed and comprises the polymorphic site of interest.

Other features and advantages of the invention will be apparent from the detailed description and from the claims.

### BRIEF DESCRIPTION OF THE DRAWINGS

FIG. 1 is a graph showing FHOD3 SNPs associated with HCM. The graph shows SNP association with HCM (−log10(observed p-value)) across the chromosome 18 locus, including the FHOD3 gene. The leading hit is the large diamond at the top of the graph. SNPs in partial linkage disequilibrium had weaker associations and are indicated by diamonds smaller in size.

FIGS. 2A and 2B are graphs of a quantitative real-time PCR (RT-PCR) analysis. FIG. 2A is a graph showing that FHOD3 expression is highest in the heart. FIG. 2B is a graph of a quantitative RT-PCR assay showing FHOD3 expression in ventricular septum and atrial appendage taken from an HCM patient at the time of surgical myectomy. FHOD3 expression is highest in the ventricle.

FIGS. 3A and 3B show that FHOD3 transcript (FIG. 3A) and protein (FIG. 3B) abundance are increased in HCM heart samples. FIG. 3A is a graph of a quantitative RT-PCR assay which shows FHOD3 transcript abundance corrected for GAPDH. Total RNA from three independent control heart samples (no HCM) and four independent HCM heart tissue samples were converted to cDNA, and quantitative RT-PCR assays were used to measure FHOD3 and GAPDH transcript levels. Significantly higher FHOD3 transcript levels were found in the HCM samples compared with the control samples. Demonstration of the FHOD3 transcript in HCM heart muscle supports an association with HCM (*, p<0.05 versus control). FIG. 3B is a Western blot of FHOD3 and a GAPDH loading control in three independent control (no HCM) heart samples compared with four independent HCM heart lysates. Demonstration of increased FHOD3 protein in HCM heart muscle supports an association with HCM.

FIG. 4 is an agarose gel of exon-specific PCR demonstrating strong expression of FHOD3 exon 10-14 in the heart. Sanger sequencing confirmed inclusion of exon 11b. GAPDH is shown as a loading control.

### DETAILED DESCRIPTION

Formin homology 2 domain containing 3 (FHOD3) polypeptide is a component of the cardiac contractile apparatus, and expression of FHOD3 is enriched in the heart. As described herein, we have identified polymorphisms of the formin homology 2 domain containing 3 gene (FHOD3) that are associated with hypertrophic cardiomyopathy. The discovery of FHOD3 gene variants associated with HCM has several clinical implications, as FHOD3 variants appear to modify the presentation and phenotype of HCM.

### Hypertrophic Cardiomyopathy

The present invention features methods for the diagnosis and prognosis of hypertrophic cardiomyopathy by detecting one or more SNPs of the formin homology 2 domain containing 3 gene. Hypertrophic cardiomyopathy is a disorder of heart muscle that is characterized by abnormal thickening or enlargement of ventricular walls, obstruction of blood flow at the left ventricular outflow tract, and myocyte disarray, resulting in systolic and diastolic dysfunction. The clinical symptoms in individuals with HCM are variable and may reflect differences in the pathophysiological manifestations of this disease. Affected individuals frequently present with exertional dypsnea, angina pectoris, or ischemia. Sudden, unexpected death is the most serious consequence of HCM and occurs in both asymptomatic and symptomatic individuals.

Additional diagnostic methods that may be used in conjunction with the methods described herein include two-dimensional echocardiography and doppler ultrasonography to quantitate ventricular wall thickness and cavity dimensions and to demonstrate the presence or absence of systolic anterior motion of the mitral valve. Electrocardiographic findings include bundle-branch block, abnormal Q waves, and left ventricular hypertrophy with repolarization changes. Other diagnostic methods include, e.g., cardiac catheterization, cardiac magnetic resonance imaging, assessing family history of HCM, and identifying disease symptoms.

### Detection of Single Nucleotide Polymorphisms (SNPs)

Methods for detecting SNPs present in a polynucleotide sequence involve procedures that are well known in the art (e.g., amplification of nucleic acids). See, e.g., Single Nucleotide Polymorphisms: Methods and Protocols, Pui-Yan Kwok (ed.), Humana Press, 2003. Although many detection methods employ polymerase chain reaction (PCR) steps to detect SNPs of a polynucleotide, other amplification protocols may also be used including, e.g., ligase chain reactions, strand displacement assays, and transcription-based amplification systems.

In general, detection of SNPs (e.g., FHOD3 SNPs) or other polymorphisms can be performed using oligonucleotide primers and/or probes. Oligonucleotides can be prepared by any suitable method (e.g., chemical synthesis). Oligonucleotides can be synthesized using commercially available reagents and instruments. Alternatively, they can be purchased through commercial sources. Methods of synthesizing oligonucleotides are well known in the art (see, e.g., Narang et al., Meth Enzymol. 68: 90-99, 1979 and U.S. Pat. No. 4,458,066). In addition, modifications to such methods of oligonucleotide synthesis may be used, e.g., to impact enzyme behavior with respect to the synthesized oligonucleotides. For example, incorporation of modified phosphodiester linkages (e.g., phosphorothioate, methylphosphonates, phosphoamidate, or boranophosphate) into an oligonucleotide may be used to prevent cleavage of the oligonucleotide at a selected site.

The genotype of an individual for an FHOD3 polymorphism can be determined using many detection methods that are well known in the art including, e.g., hybridization using allele-specific oligonucleotides, primer extension, allele-specific ligation, sequencing, or electrophoretic separation techniques, e.g., single-stranded conformational polymorphism (SSCP) and heteroduplex analysis. Exemplary assays include 5′-nuclease assays, template-directed dye-terminator incorporation, molecular beacon allele-specific oligonucleotide assays, single-base extension assays, and SNP scoring by real-time pyrophosphate sequences. Analysis of amplified sequences can be performed using various technologies such as microarrays, fluorescence polarization assays, and matrix-assisted laser desorption ionization (MALDI) mass spectrometry.

Detecting the presence of a SNP is generally performed by analyzing a sample (e.g., a biological sample containing nucleic acid) that is obtained from an individual. Often, the biological sample includes genomic DNA. The genomic DNA is typically obtained from blood samples, but may also be obtained from other cells (e.g., cardiomyocytes) or tissues (e.g., cardiac tissue). For example, the biological sample may include cells, protein or membrane extracts of cells, or blood, or biological fluids. Biological samples may be obtained by standard methods including, e.g., venous puncture and surgical biopsy. In certain embodiments, the biological sample is a cardiac tissue sample obtained by needle or surgical biopsy.

It is also possible to analyze RNA samples for the presence of polymorphic alleles. For example, mRNA can be used to determine the genotype of an individual at one or more FHOD3 polymorphic sites. In this case, the biological sample is obtained from cells in which the target nucleic acid is expressed, e.g., cardiomyocytes. Such an analysis can be performed by first reverse-transcribing the target RNA using, for example, a viral reverse transcriptase, and then amplifying the resulting cDNA or, alternatively, using a combined high-temperature reverse-transcription-polymerase chain reaction (RT-PCR), as described in U.S. Pat. Nos. 5,310,652; 5,322,770; 5,561,058; 5,641,864; and 5,693,517.

Other nucleic acid samples that may be analyzed include, e.g., genomic fragmented DNA, PCR-amplified DNA, and cDNA.

The nucleic acid samples taken from an individual may be compared, for example, to the wild-type nucleic acid sequence of, e.g., FHOD3, described herein.

Frequently used methodologies for the analysis of biological samples to detect SNPs are briefly described. However, any method known in the art can be used in the invention to detect the presence of SNPs.

### Allele-Specific Hybridization

This technique, also referred to as allele-specific oligonucleotide (ASO) hybridization, relies on distinguishing between two DNA molecules differing by one base by hybridizing an oligonucleotide probe that is specific for one of the variants to an amplified product obtained from amplifying the nucleic acid obtained from the biological sample. This method typically employs short oligonucleotides, e.g., oligonucleotides 15-20 bases in length. The oligonucleotide probes are designed to hybridize to one variant, but not to another variant. Hybridization conditions should be sufficiently stringent so that there is a significant difference in hybridization intensity between alleles, whereby an oligonucleotide probe hybridizes to only one of the alleles. The amount and/or presence of an allele may be determined by measuring the amount of allele-specific oligonucleotide that is hybridized to the sample. Typically, the oligonucleotide is labeled (e.g., with a fluorescent label). For example, an allele-specific oligonucleotide may be applied to immobilized oligonucleotides representing FHOD3 SNP sequences. After stringent hybridization and subsequent washing, fluorescence intensity is measured for each SNP oligonucleotide.

According to the invention, SNPs can be identified in a high throughput fashion via a microarray that allows the identification of one or more SNPs at any given time. Such microarrays are described, for example, in WO 00/18960. An array usually involves a solid support on which nucleic acid probes have been immobilized. These arrays may be produced using mechanical synthesis methods or light-directed synthesis methods that incorporate a combination of photolithographic methods and solid-phase synthesis methods. (See, for example, Fodor et al., Science 251: 767-777, 1991, and U.S. Pat. Nos. 5,143,854 and 5,424,186, each of which is hereby incorporated by reference.) Although a planar array surface is typically used, the array may be fabricated on a surface of virtually any shape or even a multiplicity of surfaces. Arrays may be nucleic acids on beads, fibers (e.g., fiber optics), glass, or any other appropriate substrate. In one embodiment, the microarray is a beadchip (e.g., a 370CNV Infinium chemistry-based whole genome DNA analysis beadchip (Illumina)).

In one example, SNP arrays utilize ASO hybridization to detect polymorphisms. SNP arrays include immobilized nucleic acid sequences or target sequence, one or more labeled allele-specific oligonucleotide probes, and a detection system that records and interprets the hybridization signal. To achieve relative concentration independence and minimal cross-hybridization, raw sequences and SNPs of multiple databases are scanned to design the probes. Each SNP on the array is interrogated with different probes.

Other suitable assay formats for detecting hybrids formed between probes and target nucleic acid sequences in a sample are known in the art and include the immobilized target (e.g., dot-blot) formats and immobilized probe (e.g., reverse dot-blot or line-blot) assay formats. Dot-blot and reverse dot-blot assay formats are described in U.S. Pat. Nos. 5,310,893; 5,451,512; 5,468,613; and 5,604,099, each incorporated herein by reference.

### Allele-Specific Primers

Polymorphisms are also commonly detected using allele-specific amplification or primer extension methods. These reactions typically involve use of primers that are designed to specifically target a polymorphism via a mismatch at the 3′-end of a primer. The presence of a mismatch affects the ability of a polymerase to extend a primer when the polymerase lacks error-correcting activity. For example, to detect an allele sequence using an allele-specific amplification- or extension-based method, a primer complementary to one allele of a polymorphism is designed such that the 3′-terminal nucleotide hybridizes at the polymorphic position. The presence of the particular allele can be determined by the ability of the primer to initiate extension. If the 3′-terminus is mismatched, the extension is impeded.

In some embodiments, the primer is used in conjunction with a second primer in an amplification reaction. The second primer hybridizes at a site unrelated to the polymorphic position. Amplification proceeds from the two primers leading to a detectable product, signifying the particular allelic form is present. Allele-specific amplification- or extension-based methods are described, for example, in WO 93/22456 and in U.S. Pat. Nos. 5,137,806; 5,595,890; 5,639,611; and 4,851,331.

### Detectable Probes

Genotyping can also be performed using a TaqMan (Applied Biosystems) (or 5′-nuclease) assay, as described in U.S. Pat. Nos. 5,210,015; 5,487,972; 5,491,063; 5,571,673; and 5,804,375.

The TaqMan probe principle relies on the 5′→3′ nuclease activity of Taq polymerase to cleave a dual-labeled probe during hybridization to the complementary target sequence and fluorophore-based detection. TaqMan probes consist of a fluorophore covalently attached to the 5′-end of the oligonucleotide probe and a quencher at the 3′-end. Several different fluorophores (e.g., 6-carboxyfluorescein or tetrachlorofluorescin) and quenchers (e.g., tetramethylrhodamine or dihydrocyclopyrroloindole tripeptide) may be used. The quencher molecule quenches the fluorescence emitted by the fluorophore when excited by the cycler's light source via fluorescence resonance energy transfer. As long as the fluorophore and the quencher are in proximity, quenching inhibits a fluorescence signal.

TaqMan probes are designed such that they anneal within a DNA region amplified by a specific set of primers. As the Taq polymerase extends the primer and synthesizes the nascent strand, the 5′→3′ exonuclease activity of the polymerase degrades the probe that has annealed to the template. Degradation of the probe releases the fluorophore from the probe such that the fluorophore and quencher are no longer in close proximity, thus relieving the quenching effect and allowing fluorescence of the fluorophore. Hence, fluorescence detected in, for example, a real-time PCR thermal cycler is directly proportional to the fluorophore released and the amount of DNA template present in the PCR.

The hybridization probe can be an allele-specific probe that discriminates between the SNP alleles. Alternatively, the method can be performed using an allele-specific primer and a labeled probe that binds to amplified product.

Probes detectable upon a secondary structural change are also suitable for detection of a polymorphism, including SNPs. Exemplary secondary structure or stem-loop structure probes include molecular beacons (e.g., Scorpion® primers and probes). Molecular beacon probes are single-stranded oligonucleotide probes that can form a hairpin structure in which a fluorophore and a quencher are usually placed on the opposite ends of the oligonucleotide. At either end of the probe, short complementary sequences allow for the formation of an intramolecular stem, which enables the fluorophore and quencher to come into close proximity The loop portion of the molecular beacon is complementary to a target nucleic acid of interest. Binding of the probe to its target nucleic acid of interest forms a hybrid that results in the opening of the stem loop and a conformational change that moves the fluorophore and the quencher away from each other, leading to a more intense fluorescent signal.

### DNA Sequencing and Single Base Extensions

SNPs can also be detected by direct sequencing. Methods include, e.g., dideoxy sequencing, Maxam-Gilbert sequencing, chain-termination sequencing (e.g., Sanger method), pyrosequencing, Solexa sequencing, SOLiD sequencing, or any other sequencing method known to one of skill in the art.

### Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution. Polymorphisms may also be detected using capillary electrophoresis. Capillary electrophoresis allows identification of repeats in a particular allele. The application of capillary electrophoresis to the analysis of DNA polymorphisms is well known in the art.

### Single-Strand Conformation Polymorphism Analysis

Alleles of target sequences can be differentiated using single-strand conformation polymorphism analysis, which identifies base differences by alteration in electrophoretic migration of single-stranded PCR products. Amplified PCR products can be generated as described above and heated or otherwise denatured to form single-stranded amplification products. Single-stranded nucleic acids may refold or form secondary structures, which are partially dependent on the base sequence. The different electrophoretic mobilities of single-stranded amplification products can be related to base-sequence differences between alleles of target sequences.

SNP detection methods often employ labeled oligonucleotides. Oligonucleotides can be labeled by incorporating into or onto an oligonucleotide a label detectable by spectroscopic, photochemical, biochemical, immunochemical, or chemical means. Useful labels include fluorescent dyes (e.g., fluorescein, rhodamine, Oregon green, eosin, cyanine derivatives, naphthalene derivatives, coumarin derivatives, oxadiazole derivatives, BODIPY, pyrene derivatives, proflavin, acridine orange, crystal violet, malachite green, Alexa Fluor, porphin, phtalocyanine, bilirubin, DAPI, Hoechst 33258, Lucifer yellow, or quinine), radioactive labels (e.g., ³²P), electron-dense reagents, enzymes (e.g., peroxidase or alkaline phosphatase), biotin, fluorescent proteins (e.g., green fluorescent proteins), or haptens and proteins for which antisera or monoclonal antibodies are available. Labeling techniques are well known in the art.

### SNP Detection Kits

Detection reagents can be developed and used to assay SNPs of the present invention (individually or in combination), and such detection reagents can be readily incorporated into a kit format. Accordingly, the present invention further provides SNP detection kits, including but not limited to, packaged probe and primer sets (e.g., TaqMan probe and primer sets), arrays and/or microarrays of nucleic acid molecules, and beads that contain one or more probes, primers, or other detection reagents for detecting one or more SNPs of the present invention. The kits can optionally include various electronic hardware components, containers, and devices.

### Disease Diagnosis and Predisposition Screening

An association or correlation between a genotype and disease-related phenotype (e.g., HCM) can be exploited in several ways. For example, in the case of a statistically significant association between one or more SNPs with predisposition to a disease for which treatment is available, detection of such a genotype pattern in a subject may justify immediate administration of treatment or regular monitoring of the subject.

The SNPs of the invention may contribute to HCM in a subject in different ways. Some polymorphisms may occur within a coding sequence and may contribute to disease phenotype by affecting protein structure. Other polymorphisms may occur in non-coding regions, but may exert phenotypic effects indirectly, e.g., by affecting replication, transcription, and/or translation. A single SNP may affect more than one phenotypic trait. Likewise, a single phenotypic trait may be affected by multiple SNPs in different genes.

The methods described herein may be used to diagnose a subject with, or with a predisposition to develop, hypertrophic cardiomyopathy. Such methods include, but are not limited to, any of the following: detection of HCM that a subject may presently have or be at risk for developing; predisposition screening (i.e., determining the increased risk for a subject in developing HCM in the future or determining whether an individual has a decreased risk of developing HCM in the future); determining a particular type or subclass of HCM in a subject known to have HCM; confirming or reinforcing a previously made diagnosis of HCM; following the success of a therapeutic regimen; or pharmacogenomic evaluation of a subject to determine which therapeutic strategy that subject is most likely to respond to or to predict whether a subject is likely to respond to a particular treatment. Such diagnostic uses may be based on the presence or absence of one or more SNPs (e.g., SNPs present in the FHOD3 gene (e.g., rs516514)).

Linkage disequilibrium (LD) refers to the co-inheritance of alleles (e.g., alternative nucleotides) at two or more different SNP sites at frequencies greater than would be expected from the separate frequencies of occurrence of each allele in a given population. The expected frequency of co-occurrence of two alleles that are inherited independently is the frequency of the first allele multiplied by the frequency of the second allele. Alleles that co-occur at expected frequencies are said to be in linkage equilibrium. In contrast, LD refers to any non-random genetic association between allele(s) at two or more different SNP sites, which is generally due to the physical proximity of the two loci along a chromosome. LD can occur when two or more SNP sites are in close physical proximity to each other on a given chromosome and, therefore, alleles at these SNP sites will tend to remain unseparated for multiple generations with the consequence that a particular nucleotide (allele) at one SNP site will show a non-random association with a particular nucleotide (allele) at a different SNP site located nearby. Hence, genotyping one of the SNP sites will give almost the same information as genotyping the other SNP site that is in LD.

For diagnostic purposes, if a particular SNP site is found to be useful for diagnosing HCM, then the skilled artisan would recognize that other SNP sites which are in LD with this SNP site would also be useful for diagnosing the condition. Various degrees of LD can be encountered between two or more SNPs with the result being that some SNPs are more closely associated (i.e., in stronger LD) than others. Furthermore, the physical distance over which LD extends along a chromosome differs between different regions of the genome, and the degree of physical separation between two or more SNP sites necessary for LD to occur can differ between different regions of the genome.

For diagnostic applications, polymorphisms that are not the actual disease-causing (i.e., causative) polymorphisms, but are in LD with such causative polymorphisms, are also useful. In such instances, the genotype of the polymorphism(s) that are in LD with the causative polymorphism are predictive of the genotype of the causative polymorphism and, consequently, predictive of the phenotype (e.g., HCM) that is influenced by the causative SNP(s). Thus, polymorphic markers that are in LD with causative polymorphisms are useful as diagnostic markers and are particularly useful when the actual causative polymorphism(s) are unknown.

As described herein, diagnostics may be based on a single SNP or a group of SNPs that are associated with the HCM phenotype. Combined detection of a plurality of SNPs (e.g., 2, 3, 4, 5, 6, 7, 8, 9, 10, 11, 12, 13, 14, 15, 16, 17, 18, 19, 20, 25, 30, 35, 40, 45, 50, 75, 100, or more) typically increases the probability of an accurate diagnosis. To increase the accuracy of diagnosis or predisposition screening, analysis of the SNPs of the present invention can be combined with other diagnostic methods including, e.g., echocardiography, cardiac catheterization, cardiac magnetic resonance imaging, assessing family history of HCM, and identifying disease symptoms.

### EXAMPLES

The present invention is illustrated by the following examples, which are in no way intended to be limiting of the invention.

### Example 1

### FHOD3 Gene Associated with Hypertrophic Cardiomyopathy in Genome-Wide Association Analysis

Patients of the hypertrophic cardiomyopathy (HCM) clinic at Tufts Medical Center enrolled in an institutional review board (IRB)-approved study, wherein DNA and clinical information was collected from the enrolled patients. All patients signed an informed consent form for genetic studies. DNA was collected from 178 patients diagnosed with HCM, representing 160 different families (58% male; 95.5% Caucasian; and average age of 45.9 years (ages ranging from 9 to 78 years old)). Heart structure, analyzed by echocardiography, demonstrated an average basal septal wall thickness of 19.8 mm (range of 16-51 mm) The electrocardiography studies also demonstrated that 62% of enrolled patients had either a resting or an exercise-inducible left ventricular (LV) outflow tract pressure gradient, and 31% of subjects had undergone septal reduction surgery. Blood was collected in PaxGene DNA tubes (Qiagen), and DNA was purified using a dedicated purification kit.

Genome-wide polymorphism array was performed on 125 subjects in the HCM cohort by deCode (Reykjavik, Iceland) using a 370CNV array (Illumina) Genotypes for 823 control individuals (average age of 43; ages ranging from 30 to 88 years old) obtained from Illumina iControlDB (Daw et al., Hum Mol Genet. 16(20): 2463-2471, 2007) genotyped on the same platform as our experimental study was our control cohort. All non-overlapping probes were set to “missing” for the purposes of this study, leaving ˜311,000 probes for analysis.

Polymorphisms in six chromosomal loci showed a significant difference in allele frequencies between cases and controls after correction for multiple testing.

The leading FHOD3 SNP (rs516514) was associated with an odds ratio (OR) of 2.445 (p=1.25×10⁻⁷) of HCM (Table 1). The rs516514 SNP has the following sequence. The two nucleotides in bold are the two nucleotides that define the polymorphism.

(SEQ ID NO: 1)
ATTTCCTTAATTCTATTCACATTTCAGCCCATTGCAAGTTTTTTTGTC
CTCATCTGTCATCTCATTGCCTTGTTCAATTATTCTAAAGAAGTGTAT
TGTGAGACTGATATATAGTAAAATATTTTTAGTGCATGTATAATAGAA
AATAGTTTTATAAAATCACTTTTTTTAAAATTTTAGATTTGGTGGTAC
ATGTGCTGGTTTGTTAATCGGTATCTTTCATGATGCTGAGGTTTAAGC
TTCTAATGATCCTGTTACCCAAATAATGAATGTATTACCTGATAGATA
GGTAGAAAATAGTTTTAGATTGAAAGCTGATTCTAGCT[C/T]ATCTA
CGTCTTAACATAGACCAACTGTAAAATATATATAGAATGTATATACAT
TCCTGGAATACTTCTATAACTCCCATTCTTGAGGGCTGCTTACATGTC
AAGCATGTTGCTTGCTATTTTCTGTCTGTTACCACCTTTTCTCCTGCG
AGCACACTCACAGTATGATACTTTTCCCCAATTTACAGACAAAAATCT
GAAACAAGAAGACCCGAGGCATTCAGTGATTTTCCCCAAGCATTGCCT
AGGTACCGGCAGACCCTCAATTCTAACCAAGGTCTGACTGGTGCTTAC
TTCAGTACTTTTAATCACAGGCCAAGCAACTGAAACATTAACTCATTG
TATCTCCAGGTCATCACCGAGTGGTCTTCTCACATCATCCTTCAGGCA
GCACCAAGAGTCACTGGCAGCAGAGAGAGAGAGGCGGCGGCAGGAGAG
AGAAGAA

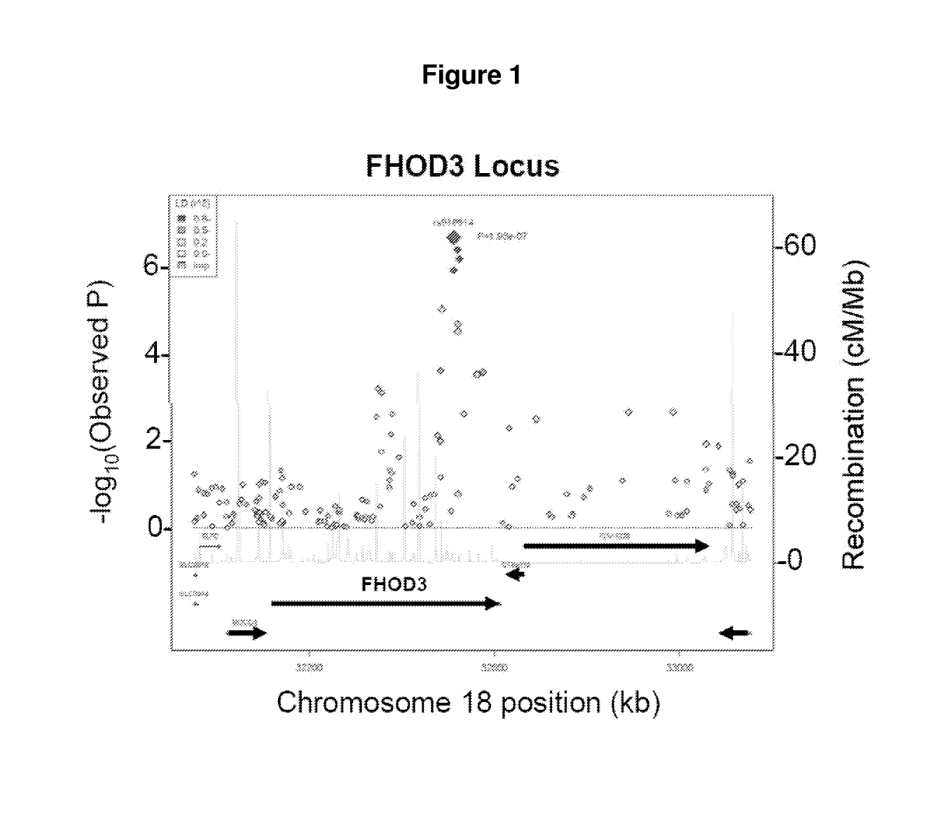

When the analysis was repeated with inclusion of and accounting for related family members, FHOD3 rs516514 was again associated with HCM (p=2.1*10⁻⁷). FHOD3 SNP polymorphisms in partial linkage disequilibrium showed weaker associations (FIG. 1). The high minor allele frequency of the FHOD3 SNP (range of 0.3-0.5 in multiple ethnic groups) indicates that the FHOD3 variant is a modifier and less likely a primary genetic cause of HCM. The association of rs516514 with HCM was also confirmed in a replication cohort (p<0.05) (Table 1). The finding of a significant association in the genome wide association cohort and replication cohort with a similar OR in both groups supports the association of this SNP with HCM.

TABLE 1
Odds ratios (OR) and p-values in genome wide association (GWA) and
replication cohorts

| | GWA | | Replication | | Composite | |
|---|---|---|---|---|---|---|
| | OR | p-value | OR | p-value | OR | p-value |
| rs516514 | 2.445 | 1.25 × 10⁻⁷ | 2.395 | 0.02868 | 2.433 | 4.91 × 10⁻⁸ |
| (FHOD3) | | | | | | |

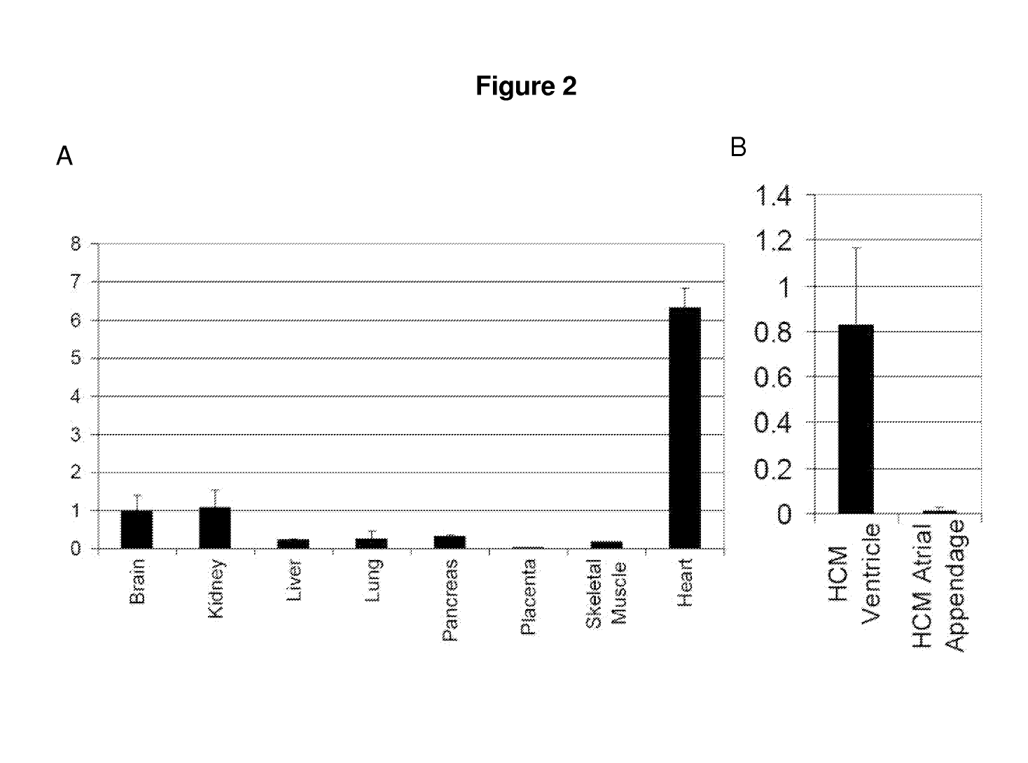

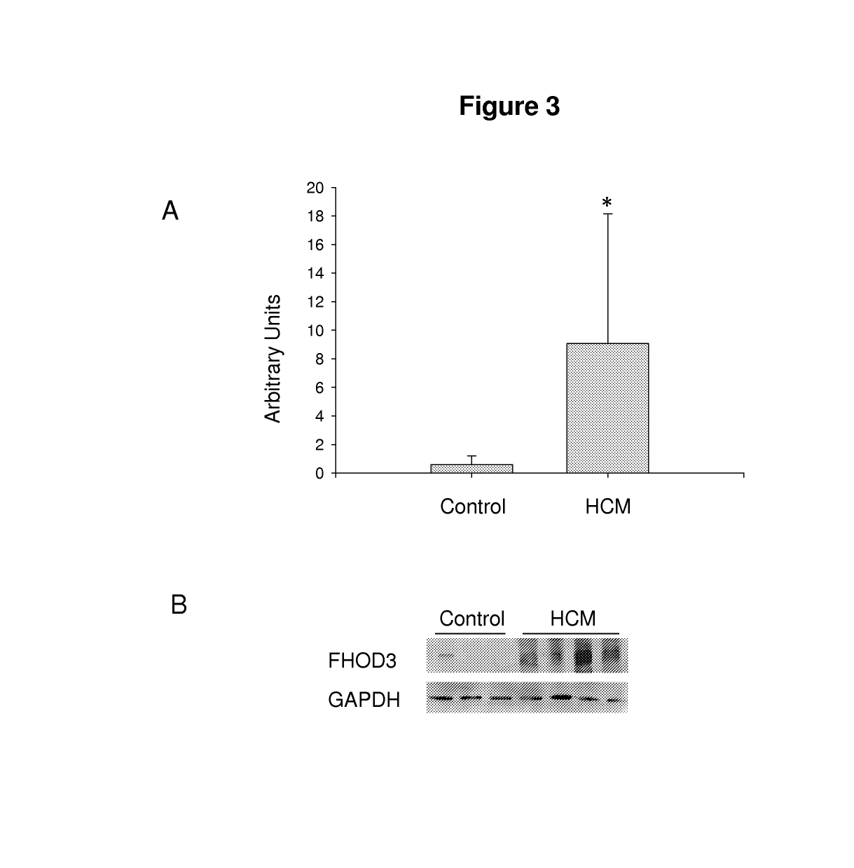

Data from the National Center for Biotechnology Information suggested strong FHOD3 expression in the heart (GDS831). Quantitative real-time PCR analysis (RT-PCR) confirmed this finding and demonstrated that FHOD3 is most strongly expressed in the heart compared with the brain, lung, kidney, and skeletal muscle (FIG. 2A). Further, FHOD3 was highly expressed in ventricular septum cardiac muscle excised from the hearts of patients with HCM at the time of septal reduction surgery; low expression in the atrial appendage tissue harvested during the same surgical procedure indicated a chamber-specific expression pattern (FIG. 2B). Quantitative RT-PCR analysis of FHOD3 transcript abundance (corrected for GAPDH) also showed significantly higher FHOD3 transcript levels in HCM samples compared with control samples (FIG. 3A), as did a Western blot analysis of FHOD3 protein levels present in the HCM and control samples (FIG. 3B). In this experiment, total RNA from three independent control heart samples (e.g., subjects not diagnosed with HCM) and four independent HCM heart tissue samples were converted to cDNA, and quantitative PCR assays were used to measure FHOD3 and GAPDH transcript levels. Demonstration of FHOD3 transcript in HCM heart muscle supports an association with HCM (*, p<0.05 versus control).

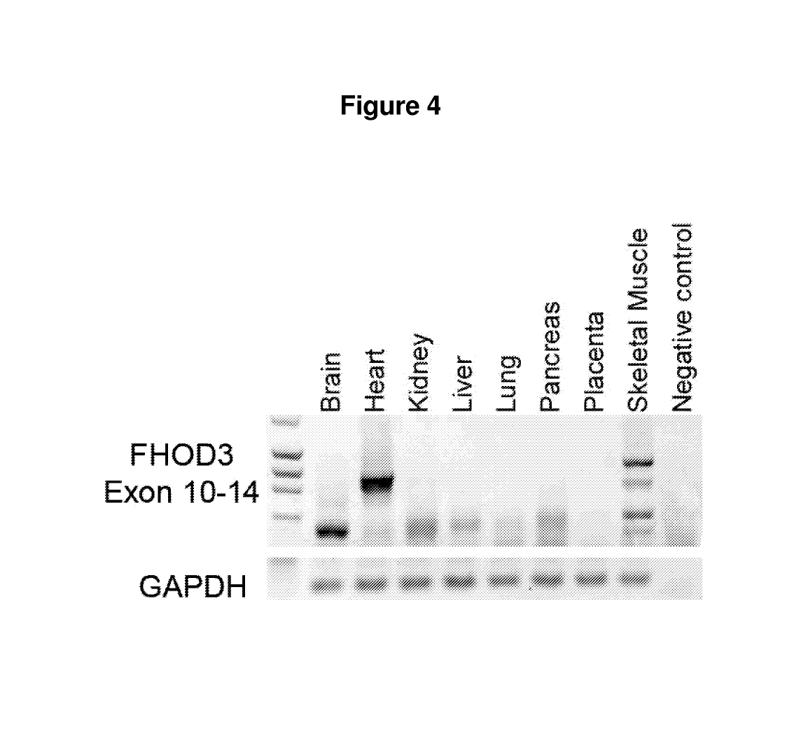

As part of the gene characterization, we performed a bioinformatics screen of FHOD3 mRNA and EST sequences deposited in NCBI. Using custom oligonucleotide primers, we indentified a novel exon 11b in heart cDNA not found in other organs (FIG. 4).

### Example 2

### Characterization of FHOD3 Variants Associated with HCM

We next perform more detailed genetic mapping and variant discovery studies on all 178 subjects in the HCM cohort.

We have identified SNPs that are in partial linkage disequilibrium (LD) with rs516514 using HapMap. Multiple synonymous and non-synonymous FHOD3 coding SNPs have been identified. The rs516514 polymorphism is in partial LD with FHOD3-I1151V (D′=0.929; r²=0.37). Because I1151V changes the FHOD3 amino acid sequence, a direct impact on the sarcomere is possible.

We perform Sanger sequencing of amplified FHOD3 exons to screen for novel variants. Novel variants that alter the amino acid sequence are expected to have the greatest impact on protein function and contractile apparatus assembly.

TaqMan assays are used to discriminate single genotypes per sample using an Applied Biosystems 7900HT Real-Time PCR machine. Bar-coded 384-well thermocycler plates are prepared with DNA, TaqMan probe, and Master Mix. Thermal cycling is performed and an end-point read is measured by the PCR machine. Genotypes assigned by an automated program are reviewed manually. Validated assays are transferred to slides for medium-throughput analysis in the BioTrove OpenArray Platform (e.g., 768 samples for 60 genotypes daily (capacity for ˜46,000 genotypes per day)). The OpenArray performs TaqMan assays on a nanoliter volume scale, saving reagent costs and speeding processing times. We genotype 10% of samples twice, as well as samples with a genotype defined by sequencing studies, to control for quality in our genotyping experiments.

For this study, we compare allele frequencies in subjects diagnosed with HCM and subjects not known to have HCM (control). HCM is defined by a maximal LV wall thickness greater than 16 mm, not explained by pressure or volume overload. Wall thickness and LV mass are quantitative variables directly correlated with the risk for adverse clinical events in HCM. Continuous measurements of maximal LV wall thickness and LV mass measured by echocardiography are analyzed against FHOD3 genotypes. Outflow tract obstruction created by contact of septal muscle with mitral valve leaflets during systole, at rest or with exercise, is also associated with an adverse outcome in HCM patients. Allele frequency differences in subjects with and without outflow tract obstruction as a categorical variable is analyzed. Outflow tract obstruction that produces severe heart failure is treated with septal surgical myectomy or catheter-based alcohol ablation. The need for septal reduction treatment is a marker for advanced HCM. Allele frequencies in subjects that received septum reduction versus those that did not are compared.

Association analysis genotype results are imported into PLINK and merged with the phenotypic data. Descriptive statistics are generated for all phenotypes. Continuous variables are presented as means (±SD) and medians (25^{th} to 75th percentiles) based on data distribution. Categorical variables are presented as proportions. For continuous variables (such as LV wall thickness), analysis of covariance is used to compare the mean scores among the three genotype groups. To account for confounding effects of variables (such as age and gender), analysis of covariance (multiple linear regression) with the ventricular endpoint as a dependent variable is employed. For a dichotomous variable, logistic regression models are used to evaluate whether differing genotypes are associated with the odds of a phenotype. Additional analyses are carried out to test for dominant, recessive, and allelic modes of inheritance.

Assuming 170 samples are available for analysis, a two-tailed significance threshold of 0.02, we have sufficient power (b>0.8) to detect a difference in LV wall thickness of half of a standard deviation unit. With a conservative recruitment estimate of an additional 30 subjects, an association at a threshold of p<0.01 can be detected.

To determine whether there is cross-talk between FHOD3 and mutant components of the sarcomere, we incorporate a multiplicative interaction term into the linear regression model. If the addition of a contractile gene mutation status creates a significant departure from the linear model of the FHOD3 SNP with LV mass, the interaction is considered significant. Likewise, a multiplicative interaction term is also employed in the logistic regression models. Finding a significant interaction between a FHOD3 SNP and contractile gene mutation status supports a role for FHOD3 variants in modifying the HCM phenotype through effects on the sarcomere.

### Other Embodiments

From the foregoing description, it is apparent that variations and modifications may be made to the invention described herein to adopt it to various usages and conditions. Such embodiments are also within the scope of the following claims.

All patents, patent applications, including U.S. Provisional Application No. 61/373,545, filed Aug. 13, 2010, and publications mentioned in this specification are herein incorporated by reference to the same extent as if each independent patent, patent application, or publication was specifically and individually indicated to be incorporated by reference.

What is claimed is:

1. A method for identifying a subject with an increased risk for developing hypertrophic cardiomyopathy (HCM), said method comprising detecting in a biological sample obtained from said subject at least one single nucleotide polymorphism (SNP) at the genomic locus of an FHOD3 gene, wherein the presence of at least one SNP at said genomic locus of said FHOD3 gene identifies said subject as having an increased risk for developing HCM.

2. The method of claim 1, wherein said SNP is rs516514.

3. The method of claim 1, wherein said biological sample comprises nucleic acid.

4. The method of claim 3, wherein said nucleic acid is one or more of DNA, genomic DNA, RNA, cDNA, hnRNA, or mRNA.

5. The method of claim 4, wherein said nucleic acid is extracted and amplified.

6. The method of claim 1, wherein said biological sample is obtained from heart tissue.

7. The method of claim 1, wherein said detecting step comprises one or more of oligonucleotide microarray analysis, allele-specific hybridization, allele-specific polymerase chain reaction (PCR), 5′ nuclease digestion, molecular beacon assay, oligonucleotide ligation assay, size analysis, or nucleic acid sequencing.

8. The method of claim 1, wherein said subject is human.

9. The method of claim 1, wherein said subject has a personal or family history of heart disease.

10. A kit comprising an assay for detecting at least one SNP at the genomic locus of an FHOD3 gene in a biological sample obtained from a subject, wherein the presence of at least one SNP at said genomic locus of said FHOD3 gene identifies said subject as having an increased risk for developing HCM.

11. The kit of claim 10, wherein said assay comprises nucleic acid probes and/or primers specific to said at least one SNP at the genomic locus of said FHOD3 gene.

12. The kit of claim 10, wherein said SNP is rs516514.

13. The kit of claim 10, further comprising instructions for correlating said assay results with said subject's risk for having or developing HCM.

14. A microarray comprising oligonucleotide probes capable of hybridizing under stringent conditions to one or more nucleic acid molecules having a single nucleotide polymorphism (SNP) at the genomic locus of an FHOD3 gene.

15. The microarray of claim 14, wherein said SNP is rs516514.
